(19) United States
(12) Patent Application Publication
(10) Pub. No.: US 2025/0099644 A1
(43) Pub. Date: Mar. 27, 2025

(54) BACTERIA-RESPONSIVE SHAPE MEMORY POLYMERS

(71) Applicants: Mary Beth Monroe, Syracuse, NY (US); Olga Makhlynets, Syracuse, NY (US); Maryam Ramezani, Syracuse, NY (US)

(72) Inventors: Mary Beth Monroe, Syracuse, NY (US); Olga Makhlynets, Syracuse, NY (US); Maryam Ramezani, Syracuse, NY (US)

(73) Assignee: Syracuse University, Syracuse, NY (US)

(21) Appl. No.: 18/576,929

(22) PCT Filed: Jul. 6, 2022
(86) PCT No.: PCT/US22/73447

§ 371 Date: Jan. 5, 2024

Related U.S. Application Data
(60) Provisional application No. 63/218,688, filed on Jul. 6, 2021.

(51) Int. Cl. A61L 15/26; A61L 15/20; A61L 15/42; C08L 71/02; C08L 77/04

(52) U.S. Cl. CPC A61L 15/26; A61L 15/20; A61L 15/42; C08L 71/02; C08L 77/04; A61L 2300/254

(57) ABSTRACT

A bacteria-responsive shape memory polymer (SMP) platform with clinical impact for chronic wound infection control. A bacteria-responsive SMP is synthesized in a primary shape and then fixed in a secondary shape until exposed to a second stimulus, which comprises the presence of bacterial proteases. During active infection, bacteria release proteases that degrade peptides in the SMP backbone and induce recovery to the primary shape, thereby providing a visual indication of an infection. In addition, on the microscopic scale, the SMP shape change can help inhibit biofilm formation and make them more susceptible to treatment with antibiotics.

### CROSS-REFERENCE TO RELATED APPLICATIONS

The present application claims priority to U.S. Provisional App. No. 63/218,688, filed on Jul. 6, 2021.

### BACKGROUND OF THE INVENTION

### 1. Field of the Invention

The present invention related to medical devices and, more specifically, to wound dressings that can provide a visual indication of a bacterial infection and inhibit bacterial colonization.

### 2. Description of the Related Art

Chronic wounds, including diabetic ulcers and bed sores, affect more than 5 million patients per year in the US alone. The cost of healing one diabetic ulcer is estimated to be $50,000, and diabetic ulcers are associated with approximately 70 percent of foot amputations. Of the many factors that delay healing in chronic wounds, infection plays a significant role, and antibiotic resistance renders this problem increasingly difficult to manage. To further complicate this clinical need, about 50 percent of diabetic ulcer patients with a limb threatening infection do not show systemic infection symptoms, thereby delaying diagnosis and treatment.

Despite the prevalence of chronic wound infections, there is no expert consensus on the standard of care for assessment and treatment. Currently, infection assessment requires wound swabbing, which increases wound exposure to potential pathogens in the environment. Additionally, swabs can be easily contaminated with skin surface bacteria, and they may not effectively penetrate biofilms, both of which can contribute to misdiagnoses. Treating infections typically involves painful debridement and the use of local antimicrobials, such as iodine or silver, which could hinder healing. Furthermore, about 60 percent of chronic wounds contain biofilm (vs. 6 percent of acute wounds), which is less amenable to standard antimicrobial treatment. Thus, there remains a critical need for chronic wound dressings that specifically respond to active infections to aid in earlier surveillance and treatment and, in the absence of improved options, infections will remain a critical hindrance in chronic wound healing.

### BRIEF SUMMARY OF THE INVENTION

The present invention is a smart biomaterial system that specifically responds to infection to aid in surveillance and treatment. The biomaterial system is based upon shape memory polymers (SMPs) that are synthesized in a primary shape. An external stimulus (e.g., heat) is applied to soften the material and allow it to be strained into a secondary shape. Upon removal of the stimulus, the secondary shape is fixed until the material is exposed to a second stimulus, upon which it returns to its primary shape. In the system of the present invention, the second stimulus is the presence of bacterial proteases. Bacteria-responsive SMPs may be applied to wounds in their secondary shape, which will remain stable unless bacteria are present.

During an active infection, bacteria will release proteases that degrade peptides in the SMP backbone and induce recovery to the primary shape. On the microscopic scale, SMP shape change can dislodge attached biofilms and make them more susceptible to treatment with antibiotics. On the macroscopic scale, this shape change would be visible to wound care clinicians, thereby alerting the clinicians to an infection and allowing them to proceed with standard treatments, which may be more effective against the dislodged bacteria.

In one embodiment, the present invention is a wound dressing formed from a shape memory polymer having a soft block bound to a bacterial protease substrate and a hard block polymerized with the soft block so that the shape memory polymer has a first shape and can be deformed into a second shape. The shape memory polymer will transition from the second shape to the first shape in the presence of a bacterial protease corresponding to the bacterial protease substrate of the shape memory polymer. The bacterial protease substrate may be SEQ ID NO: 1. The bacterial protease that is targeted may be V8. The first shape may have a first diameter and the second shape may have a second diameter that is different than the first diameter. The second diameter may be larger than the first diameter. The soft segment may be formed from poly(propylene glycol). The hard segment may be formed from hexamethylene diisocyanate and triethyelene glycol. The shape memory polymer may further comprise poly(glutamic acid). The shape memory polymer may further comprise a phenolic acid.

In another embodiment, the invention may be a method of identifying a bacterial infection that begins with the step of providing would dressing formed from a shape memory polymer having a soft block bound to a bacterial protease substrate and a hard block polymerized with the soft block. Next, the wound dressing is deformed from a first shape into a second shape. The wound dressing is then applied to a wound and monitored to determine if the wound dressing has transitioned from the second shape to the first shape, thereby indicating the presence of the bacterial protease.

### BRIEF DESCRIPTION OF THE SEVERAL VIEWS OF THE DRAWING(S)

The present invention will be more fully understood and appreciated by reading the following Detailed Description in conjunction with the accompanying drawings, in which:

FIG. 1 is a schematic showing a bacteria-responsive shape memory polymer system according to the present invention for invention surveillance and treatment:

FIG. 2 is a chart of the thermal, mechanical, and degradation properties of shape memory polymers:

FIG. 3 is a graph and associated images of the shape stability of shape memory polymers in an oxidative degradation media:

FIG. 4 is a graph and associated images of the shape stability of shape memory polymers in a hydrolytic degradation media:

FIG. 5 is a graph and associated images of the shape change of bacteria-responsive shape memory polymers in bacterial enzyme (V8) media with shape stability in oxidative and hydrolytic media; and

FIG. 6 is a schematic of shape memory polymer formulations according to the present invention.

FIG. 7 is a graph of shape change of bacteria-responsive shape memory polymers in the presence of E. coli, S. aureus, and S. epi showing that the materials are stable in the presence of mammalian fibroblasts.

FIG. 8 is images of shape memory polymer shape changes in the presence of bacteria, resulting biofilm inhibition on the surface, and increased susceptibility of attached bacteria to applied antibiotics. Control panels include the shape memory polymer applied in its first shape only in the second row, which does not affect bacteria interactions. The control materials in the third and fourth row do not have a bacteria-responsive component and also do not affect bacteria interactions.

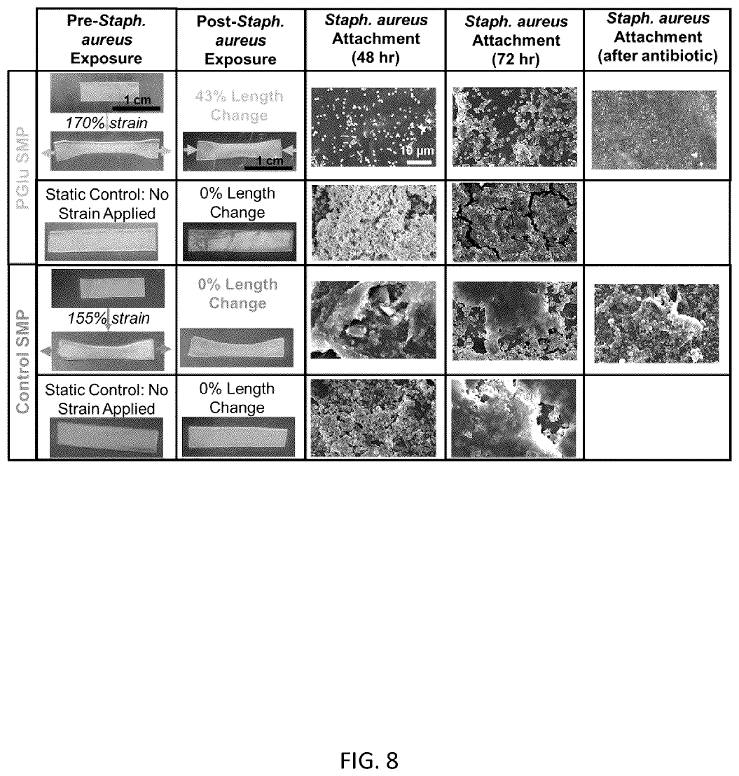

### DETAILED DESCRIPTION OF THE INVENTION

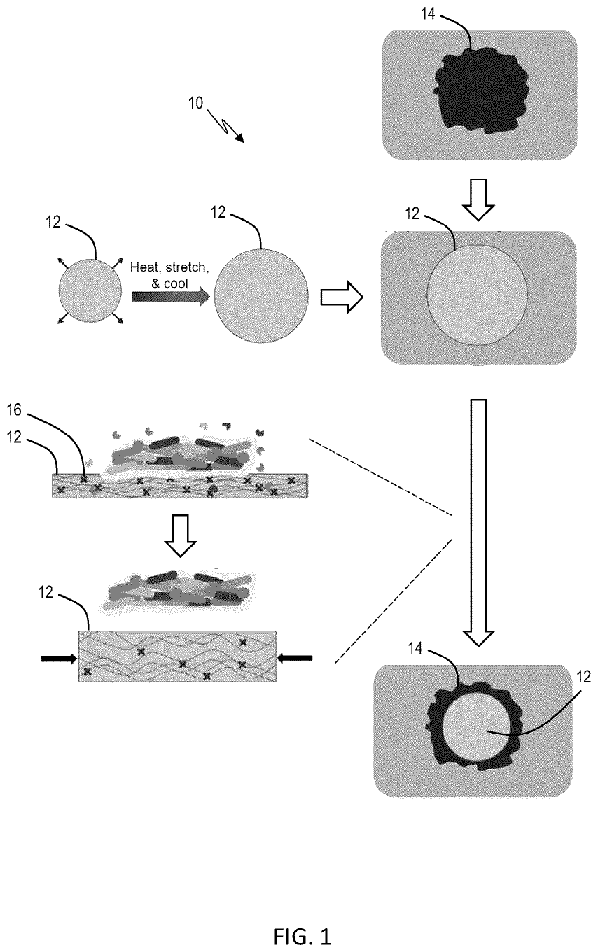

Referring to the figures, wherein like numerals refer to like parts throughout, there is seen in FIG. 1 a bacteria-responsive shape memory polymer system 10 according to the present invention for surveillance and treatment of bacterial infections. As seen in FIG. 1, bandage 12 formed from a shape memory polymer including a bacterial protease peptide target 16 according to the present invention is heated, stretched and then cooled. The peptide target has a high specificity to a bacterial protease that is commonly found in infected wounds and includes a well-defined sequence with information on relative degradation rates. The stretched bandage 12 is then applied over a wound 14. In the event of a bacterial infection in wound 14, such as Staph. aureus, peptide target 16 in bandage 12 will be degraded in response the release of proteases by the bacteria, such a V8. The degrading of peptide target 16 of bandage 12 by proteases induces a shape change in bandage 12. The shape change of bandage 12 can provide a visible indicator to a clinician of the bacterial infection, and can assist with treatment by physically dislodging bacteria from wound 14.

As an example of the present invention for use in connection with one common bacterial infection, Staph. aureus strain V8 protease is prevalent in chronic wounds worldwide, and it is associated with polymicrobial infections, making it a useful bacterial enzyme for the design of a target to be incorporated into the shape memory polymer of the present invention. The V8 protease is a serine endopeptidase that cleaves peptide bonds at the carboxyl side of Glu. It has been identified as one of the most important secreted enzymes in infected wounds. A range of peptide targets have been identified that degrade in the presence of the V8 enzyme, and their degradation rates have been established. For the present invention, the peptide sequence SAFAFEVFYDS (SEQ ID NO: 1), which is cleaved between E and V (EV peptide) and poly(glutamic acid), was selected. These sequences can be degraded within <3 hr when exposed to V8 at levels as low as 0.001-1 mg/ml.

Previous experiments have shown that shape recovery of SMPs can be induced by the presence of enzymes (thus far, by non-specific hydrolysis of ester bonds by lipase). The peptide, such as SAFAFEVFYDS (SEQ ID NO: 1) or poly(glutamic acid), is incorporated into a SMP backbone to provide network disruption and shape recovery in the presence of the targeted protease, e.g., V8 produced by Staph. aureus. The shape recovery is macroscopically visible, thereby enabling easier infection surveillance. To add in antimicrobial functionality to the polymer structure, phenolic acids (PAS) may be employed as non-drug-based antimicrobials that reduce overall infection risks. PAs contain a carboxylic acid group on their non-active end, which enables their facile incorporation into polyurethanes. Plant-based PAs are part of the plant antimicrobial mechanism, and they have broad antimicrobial efficacy against drug-resistant bacteria strains and biofilms. Their incorporation into bacteria-responsive SMPs may aid in reducing overall infection risks.

In an experiment, thermally-induced shape recovery of SMPs reduced Staph. aureus biomass from 5.4 to 0.07 μm3/μm2 (vs. no change in biomass on static sample). In further studies with the same thermo-responsive system, shape recovery with subsequent application of an antibiotic (50 μg/mL tobramycin) reduced dispersed biofilm cell counts by >3 logs (2,479-fold) in comparison to the static control sample. The observed effects were attributed to the physical disruption of the biofilm structure upon shape change that increased cellular activity. The shape recovery that occurs upon EV peptide cleavage in bacteria-responsive SMPs is likely to dislodge attached bacteria and thus assist with treatment by making the bacteria more susceptible to drug treatment in vitro and in vivo.

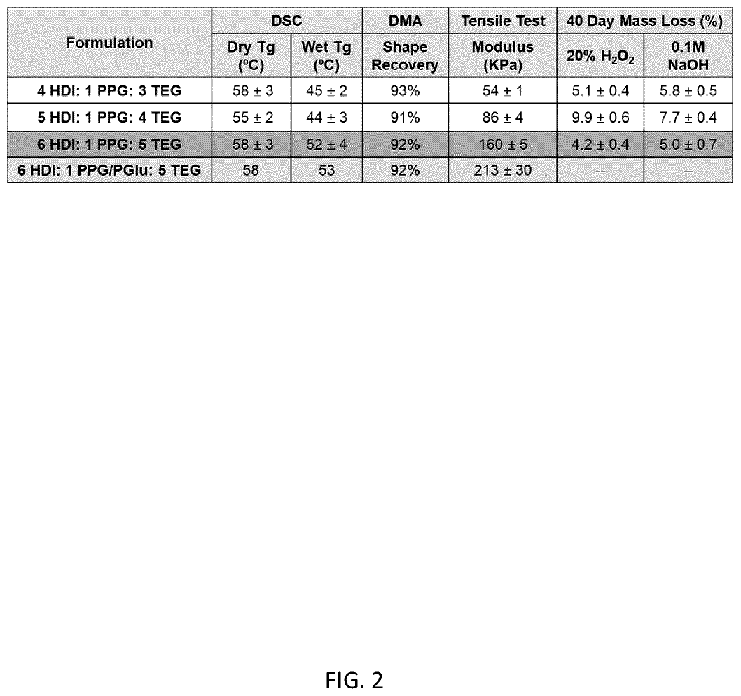

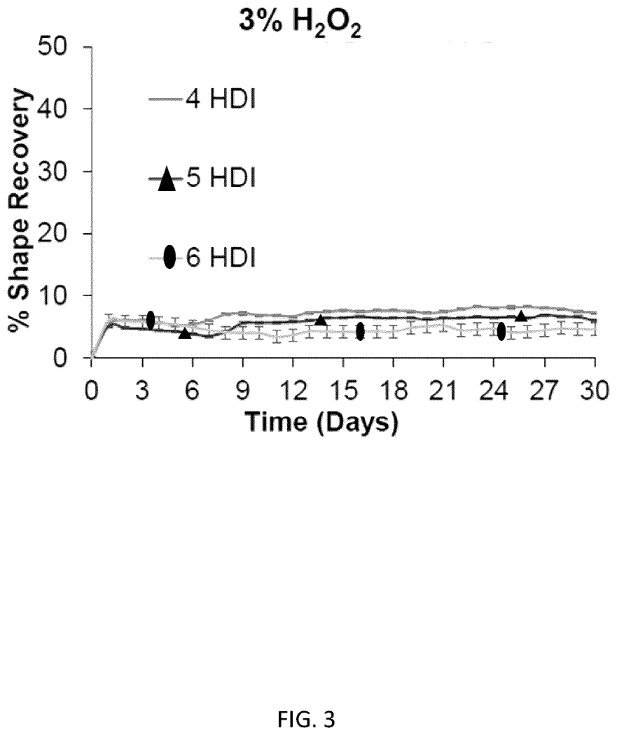

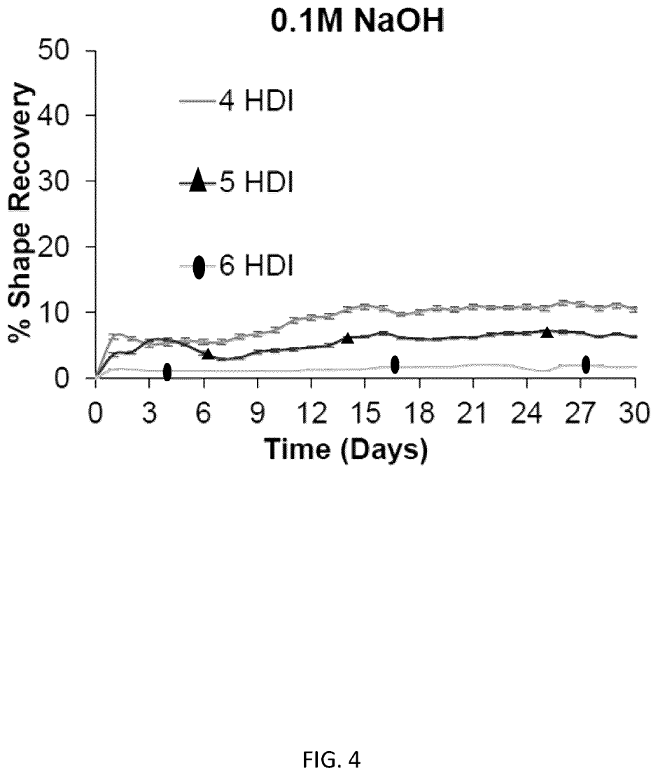

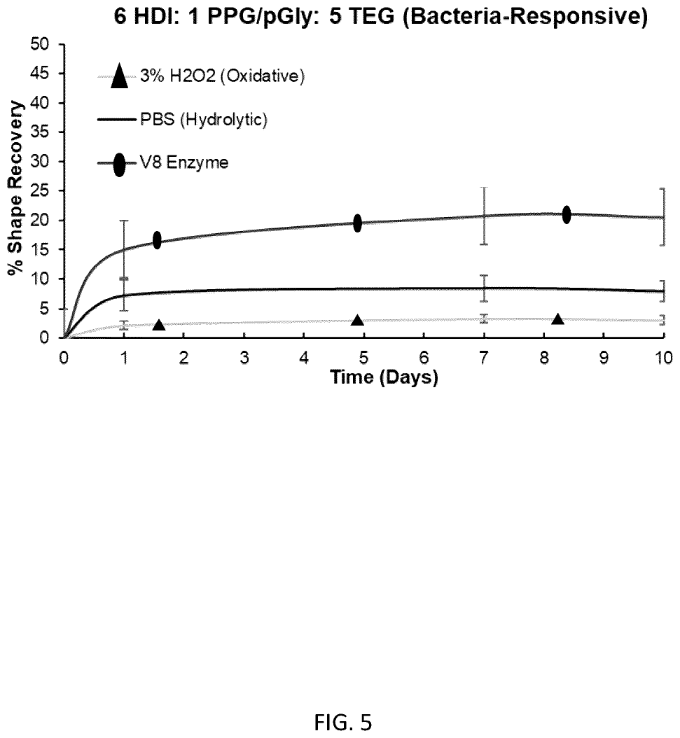

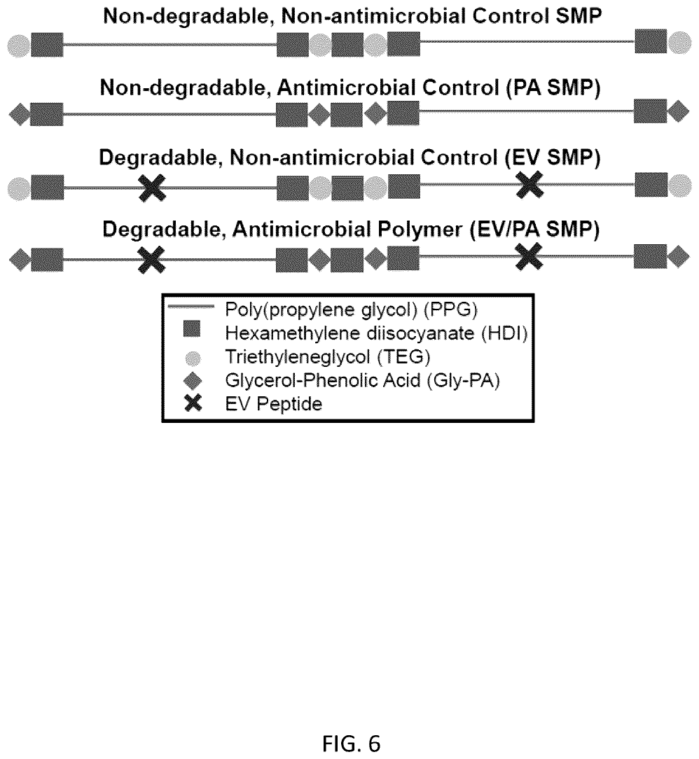

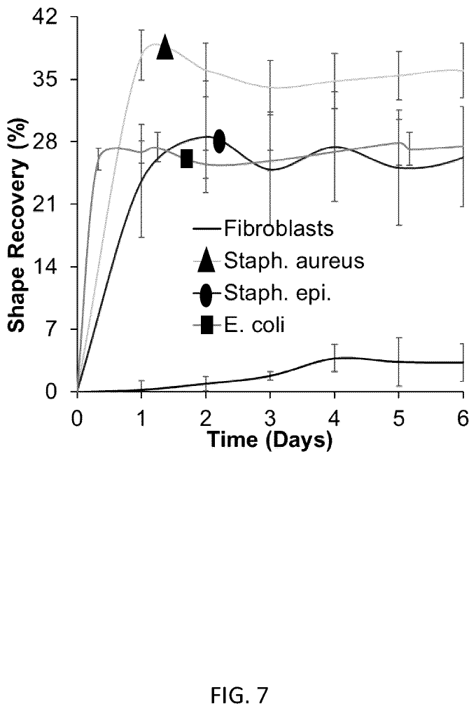

A library of control segmented polyurethanes (SPUs) was synthesized with varied ratios of soft segment (poly(propylene glycol) (PPG)) to hard segment (hexamethylene diisocyanate (HDI) and triethyelene glycol (TEG)), as seen in FIG. 2. A bacteria-responsive polymer was synthesized using poly(glutamic acid) (pGlu) as well. Tensile modulus in the control polymers ranges from 54 to 160 kPa, based upon hard segment content. Mechanical properties can be easily tuned by altering monomer ratios (increased HDI and TEG increases strength and modulus). The addition of pGlu increased modulus from 160 to 213 kPa. The control polymers all have high hydrolytic and oxidative stability, with <10% mass loss after 40 days in accelerated media (20% H2O2 or 0.1 M NaOH) at 37° C. All SPUs demonstrate shape memory with shape recovery >90%, and their dry and wet glass transition temperatures are above body temperature, as seen in FIG. 2. As an initial measure of their shape stability, samples were heated and folded them in half to lock in a secondary shape. Samples were incubated in oxidative (3% H2O2) or hydrolytic (0.1M NaOH) media at 37° C. for 30 days, and the distance between the folded sample ends was measured as shape recovery. All polymers remained folded with <10% shape recovery throughout the study, as seen in FIGS. 3 and 4. This result indicates that control SMPs would have high secondary shape stability after implantation and would therefore not change their dimensions if applied as a wound bandage in their secondary geometry. These polymers provide a platform for the controlled introduction of protease-responsive segments into the polymer backbone that specifically respond to the wound environment. The pGlu containing polymer was folded into a secondary shape and incubated in 3% H₂O₂ (oxidative media), phosphate buffered saline (PBS, hydrolytic media), or 0.1 mg V8 protease/ml PBS for 10 days. The samples showed a visible unfolding in the V8 enzyme media while remaining stable in 3% H₂O₂ and PBS, as seen in FIG. 5. In further studies, the pGlu SMPs unfolded in the presence of bacteria (E. coli, S. aureus, and S. epi.) while remaining folded in the presence of mammalian fibroblasts, as seen in FIG. 6. The shape change of pGlu SMPs in the presence of S. aureus is visible and it inhibits biofilm formation on the surface to increase bacteria susceptibility to antibiotics, as seen in FIG. 7. The pGlu SMP with the same chemistry that was incubated with bacteria in its first shape did not affect biofilm formation, indicating that the biofilm inhibition mechanism lies in the shape change. This result shows the feasibility of the proposed approach for providing materials that selectively change shape in the presence of bacterial proteases to reduce biofilm formation and make bacteria easier to treat with applied antibiotics.

### Example

Peptide synthesis: The target peptide, SAFAFEVFYDS (SEQ ID NO: 1) (EV peptide), may be synthesized using manual Fmoc solid-phase synthesis at elevated temperature with Amide Rink and Fmoc-protected amino acids as previously described. The N-terminus will be acetylated, peptides will be cleaved from the resin, and side chains will be deprotected using trifluoroacetic acid (TFA)/water/triisopropyl silane (TIS) (95:2.5:2.5 vol %). The peptide will be precipitated with cold methyl-tert-butyl ether and purified using reverse-phase high-performance liquid chromatography (HPLC). Peptide identity and purity will be assessed using MALDI-TOF mass spectrometry and HPLC to obtain peptides that are at least 90% pure.

Peptide Stability: Peptide stocks will be prepared in water, lyophilized, and stored at −20° C. The peptide and proteases will be dissolved in buffer (2 mM peptide with 0.1 μg/ml V8 enzyme or 100 U/ml MMP-1). In a separate study, Staph. aureus or human dermal fibroblasts will be seeded into Transwell inserts, and the peptide will be dissolved in media in the wells below. Peptide digestion will be tracked at 1, 2, 4, 8, 24 and 48 hr using analytical HPLC. The EV peptide is expected to degrade in the V8 solution within <4 hr and to be stable in the MMP-1 solution.

If there are issues with peptide synthesis/purification, custom peptides may be purchased to enable progress until synthesis protocols are finalized. The literature indicates that the EV peptide should only degrade in the presence of the V8 enzyme from Staph. aureus. However, if the peptide is not stable in mammalian enzymes, the following as alternatives may be tried: poly-L-glutamic acid (pGly, V8 substrate), ALA (Pseudomonas LasB substrate), or AGLA (Pseudomonas elastase substrate).

SMP Synthesis: The peptide-containing soft segment may be synthesized by reacting poly(propylene glycol) (PPG, 1000 g/mol), hexamethylene diisocyanate (HDI), and the EV peptide at a 2:2:1 ratio in tetrahydrofuran (THF) with 5% dibutyltin dilaurate (DBTDL) as a catalyst. The reaction will be monitored using Fourier transform infrared (FTIR) spectroscopy. The final product will be precipitated in cold ether and characterized using nuclear magnetic resonance (NMR) spectroscopy. The resulting EV-PPG soft segment will be reacted with 5 mol. eq. HDI in THF with 5% DBTDL. Reaction completion will be confirmed using FTIR. Then, 4 mol. eq. of Glycerol-phenolic acid (Gly-PA) may be added. FTIR will be utilized to track reaction completion, after which the final product will be precipitated in cold ether, dried under vacuum, and analyzed with NMR. The polymer will be characterized using differential scanning calorimetry (DSC: glass transition (Tg) and melting (Tm) temperatures), dynamic mechanical analysis (DMA: shape fixity and recovery), and Instron (tensile modulus, ultimate elongation, ultimate tensile strength). A non-degradable, non-antimicrobial control will be synthesized using PPG (2 kDa) as the soft segment and triethylene glycol (TEG) in place of Gly-PA (Control SMP). A non-degradable, antimicrobial control will be synthesized using PPG (2 kDa) as the soft segment with Gly-PA as the chain extender (PA SMP). A degradable, non-antimicrobial control will be synthesized using triethylene glycol (TEG) in place of Gly-PA (EV SMP). Schematics of all 4 proposed SMP formulations are shown in FIG. 6.

SMP Stability: SMP films will be prepared via solvent casting, and ASTM dogbones will be cut from each formulation. Samples will be heated to above their Tg, stretched lengthwise by 50%, and cooled to room temperature. Then, their dimensions will be measured, and they will be placed in degradation media (accelerated hydrolytic: 0.1M NaOH; accelerated oxidative: 20% H₂O₂; bacterial enzymatic: 0.1 μg/ml V8 enzyme; or mammalian enzymatic: 100 U/ml MMP-1) at 37° C. for up to 40 days. An additional set of samples will be placed in Transwell inserts with Staph. aureus or HDFs in media in the lower wells to assess indirect bacteria and cell interactions over 1 week. Each day, samples dimensions will be measured to assess shape stability. Every week, surface chemistry (FTIR), Tg (DSC), and surface morphology (scanning electron microscopy, SEM) will be assessed. Bacterial and cell viability will be measured using Live/Dead assay kits.

If peptide incorporation negatively affects SMP properties, it is possible to (1) alter hard segment content (increased HDI and Gly-PA will increase modulus and strength), (2) change soft segment length (increased PPG molecular weight will decrease modulus and strength and increase Tg), and/or (3) incorporate the peptide into the hard segment instead of the soft segment. If the shape change of EV-containing SMPs is too slow in the bacterial protease solution (i.e. >1 day), the synthesis protocols may be altered to increase peptide concentration and/or reduce SMP Tg by reducing hard segment content.

Direct Bacterial Interactions: SMP films will be prepared, and 4 mm diameter discs will be cut from each formulation (Control, PA, EV, and EV/PA SMP). The discs will be heated, stretch uniaxially, and cooled to lock in a secondary oval geometry. Staph. aureus will be incubated on the surface of the films for up to 1 week. At set time points (1, 12, 24, 48, 72, and 168 hr), a LIVE/DEAD BacLight Bacterial Viability assay will be utilized to characterize bacterial viability. Colony forming units (CFUs) will be measured via culture of droplets on LB-agar plates as an additional measurement. Confocal laser scanning microscopy (CLSM) will be utilized to image stained biofilms in 3D to quantify biofilm thicknesses. Each day, sample dimensions will be measured to determine whether shape change occurred. Using the data from this study, a second study will be carried out in which biofilms will be cultured on SMP samples. If a visible SMP shape change occurs in any sample subset, antibiotics will be applied to the media of all samples, and bacteria viability will be measured. To characterize function in the case of active infection at the time of application, Staph. aureus biofilms will be grown on LB agar plates. SMP samples in their secondary shape will be placed on top of biofilms. Sample geometries and bacterial viability will be measured over 7 days. The Control SMP and PA SMP should have no significant changes in dimension during culture. EV SMP and EV/PA SMP should undergo significant shape changes during culture, which would result in increased antibiotic susceptibility of biofilms. The PA SMP and EV/PA SMP should reduce bacterial growth compared with Control SMP and EV SMP.

Bacteria-Responsive SMP In Vivo Characterization: Adult mice will be anesthetized using inhalation isoflurane, and their backs will be shaved to prepare for injury. Full thickness excisional punch wounds (4/mouse) will be made using a 4 mm biopsy punch. Wounds will be splinted with adhesive silicone rings (5 mm ID, 9 mm OD) to reduce skin contraction. For infected wounds, Staph. aureus biofilms will be pre-formed on LB agar plates and applied at the time of wounding. Treatment materials may be applied, and a Tegaderm cover added. SMP dressing and wound dimensions will be imaged and measured daily for up to 12 days, and new dressings will be applied every 3 days. In half of the infected animals, oxacillin will be applied 12 hours before dressing changes. At each time point, wound swabbing and culture will be carried out to characterize wound bacterial burden. Then, full-thickness tissue will be harvested and stained with hematoxylin and eosin (H&E) for general histological analysis of healing and with Gram's stain or BODIPY to evaluate bacterial content. The Control SMP and PA SMP should not have any significant changes in dimension after implantation. EV SMP and EV/PA SMP should undergo significant shape changes after implantation in infected wounds but be stable in control wounds. EV SMP should increase bacterial susceptibility to applied antibiotics, but not reduce bacteria load on its own. PA SMP should decrease bacterial numbers in wounds. EV/PA SMP should reduce bacterial numbers and increase bacterial susceptibility to applied antibiotics. If the EV/PA SMP+antibiotic does not effectively eradicate infection in vitro or in vivo, methods for altering surface properties may be explored as these have been shown to increased infection control efficacy.

The present invention provides a novel bacteria-responsive SMP platform with potential clinical impact in chronic wound infection control. Bacteria-responsive SMPs could also be utilized as infection-resistant medical device coatings. The platform material system could be used to mechanistically study how bacteria interact with biomaterials, and it could be built upon by the addition of new functionalities to aid in healing, such as controlled release of growth factors or other drugs. Upon establishing a proof-of-concept with the EV peptide for shape change actuation, SMPs could be modified with a range of mammalian protease targets to provide smart materials for controlled drug delivery and regenerative medicine applications.

What is claimed is:

1. A wound dressing, comprising:
a shape memory polymer having a soft block bound to a bacterial protease substrate and a hard block polymerized with the soft block so that the shape memory polymer has a first shape and can be deformed into a second shape;
wherein the shape memory polymer will transition from the second shape to the first shape in the presence of a bacterial protease corresponding to the bacterial protease substrate of the shape memory polymer.

2. The wound dressing of claim 1, wherein the bacterial protease substrate is SEQ ID NO: 1.

3. The wound dressing of claim 2, wherein the bacterial protease is V8.

4. The wound dressing of claim 1, wherein the first shape has a first diameter and the second shape has a second diameter that is different than the first diameter.

5. The wound dressing of claim 4, wherein the second diameter is larger than the first diameter.

6. The wound dressing of claim 5, wherein the soft segment is formed from poly(propylene glycol)

7. The wound dressing of claim 6, wherein the hard segment is formed from hexamethylene diisocyanate and triethyelene glycol.

8. The wound dressing of claim 7, wherein the shape memory polymer further comprises poly(glutamic acid).

9. The wound dressing of claim 8, wherein the shape memory polymer further comprises a phenolic acid.

10. A method of identifying a bacterial infection, comprising:
providing a wound dressing formed from a shape memory polymer having a soft block bound to a bacterial protease substrate and a hard block polymerized with the soft block;
deforming the wound dressing from a first shape into a second shape;
applying the wound dressing to a wound; and
viewing the wound dressing to determine if the wound dressing has transitioned from the second shape to the first shape as a result of the presence of a bacterial protease corresponding to the bacterial protease substrate of the shape memory polymer.

11. The method of claim 10, wherein the bacterial protease substrate is SEQ ID NO: 1.

12. The method of claim 11, wherein the bacterial protease is V8.

13. The method of claim 10, wherein the first shape has a first diameter and the second shape has a second diameter that is different than the first diameter.

14. The method of claim 13, wherein the second diameter is larger than the first diameter.

15. The method of claim 14, wherein the soft segment is formed from poly(propylene glycol)

16. The method of claim 15, wherein the hard segment is formed from hexamethylene diisocyanate and triethyelene glycol.

17. The method of claim 16, wherein the shape memory polymer further comprises poly(glutamic acid).

18. The method of claim 17, wherein the shape memory polymer further comprises a phenolic acid.

19. The method of claim 10, further comprising the step of treating the wound with an antibiotic if the wound dressing has transitioned from the second shape to the first shape.


SEQUENCE LISTING (1 sequence)

SEQ ID NO: 1 - protein, 11 aa, synthetic construct
SAFAFEVFYDS